(19) United States
(12) Patent Application Publication
(10) Pub. No.: US 2019/0246984 A1
(43) Pub. Date: Aug. 15, 2019

(54) INSTRUMENTED RETRIEVABLE IMPLANTABLE DEVICE

(71) Applicant: Raymond MOSER, Engelburg (CH)

(72) Inventor: Raymond MOSER, Engelburg (CH)

(21) Appl. No.: 16/311,032

(22) PCT Filed: Jul. 6, 2016
(86) PCT No.: PCT/IB2016/054043

§ 371 Date: Dec. 18, 2018

(51) Int. Cl. A61B 5/00; A61B 5/07

(52) U.S. Cl. CPC A61B 5/6876; A61B 5/076; A61B 5/6862; A61B 5/1076; A61B 5/6886; A61B 5/4839; A61B 5/6859

(57) ABSTRACT

Instrumented retrievable implantable device (2), comprising an expandable section, for placement inside a body lumen, preferably in a blood vessel. The shape of the device of the invention (2) allows repositioning and retrieval by micro-invasive methods, by means of a link section (20) coupleable with a grabbing device (45) mounted on a catheter (40). A special positioning arm (64, 66) allows safe anchoring of the implant relative to a branching or another anatomy feature, and, the device may include soft inserts to streamline its body and reduce deposits.

### FIELD OF THE INVENTION

The present invention concerns, in embodiments, an implantable device for retrievably securing the device inside a body lumen.

### DESCRIPTION OF RELATED ART

Vascular implantable devices have long been applied, in particular in conjunction with balloon angioplasty, to restore proper flow in constricted blood vessels. According to this technique, the blood vessel is expanded with an inflatable balloon, guided into the desired section of the vessel by means of a catheter. The intravascular device or stent is then positioned inside the vessel to ensure that it maintains the enlarged diameter once the expanding balloon is removed. Often, the stent is mounted on the expandable balloon before the implant, expanded at the intended location by the balloon, and left in place

On the other hand, it is known to use miniaturized devices for performing diagnostic or research measurement inside the body of a human or an animal. Such devices are in general inserted inside a blood vessel or another body lumen by a suitable catheter and stay attached by a wire or cannula to an external monitoring device. In these procedures, the patient has a cannula to access the position, be it in a vessel, artery, or vein, at which the parameter is measured.

This procedure, even if it is only moderately invasive, requires a medical establishment and does not allow prosecuting the measurement for an extended period of time or during patient's normal activities. Cannulated patients are at risk of complications like infection, migration, disconnection, and bleeding and are physically connected to a monitoring device; they must be monitored by medical staff and their movements and activities are of necessity severely restricted. This restricts the practical application of these diagnostic devices to patients that would be in any case bound to a bed and constantly monitored. Nevertheless, other patients, whose conditions do not require intensive care hospitalization, would benefit from the accurate measurements that can be achieved only by placing a sensor directly in the target body lumen. Direct intravascular measurement, for example, is considered the “golden standard” technique for measuring blood pressure, for example. There is therefore a need of an implantable diagnostic device that can be applied to a larger cohort of patients.

Moreover, the known vascular devices are in general suitable for definitive implantation only. While safe and reliable procedures to position a vascular implant into a body lumen are well established, this is not generally true for recovering or repositioning an already implanted device. Once a device is deployed, its emplacement is considered definitive and recovering or replacing it would often require invasive surgery.

Some repositionable and recoverable vascular devices have been proposed in the past and, in particular, reference is made to U.S. Pat. Nos. 7,801,626 and 8,103,361, in the name of the inventor, which describes an implantable vascular and intraluminal device that can be positioned, repositioned, or recovered by a catheter. Similar or generally related endeavours are known from U.S. Pat. No. 4,886,065, 5,282,845, EP0553580A1, U.S. Pat. No. 5,954,761, US2002035331, US2002128546.

Some transcatheters heart valves are known, for example by US20160067041, US20140324161, US20130268064, with features for repositioning the valve during the implantation, for example to adjust their alignment and position. However, they are designed for permanent implant and should not be removed once the implant procedure is complete.

Manipulating intravascular and intraluminal devices by catheter techniques requires, in general, the provision of special features on the device that are designed to cooperate with prehensile implements attached to the catheter. Such features, for example knobs, holes, or hooks, allow for a firmer connection with the prehensile implements of the catheter but, on the other side, might be a site of accumulation of deposits of various nature. It is known that intravascular device or temporary catheterisations are linked to increase risk of thrombosis, or particle formation leading to ischemia, etc. For permanent implanted devices, tissue growths are observed around the parts in direct contact with endothelium. After an extended time from implantation, the device can be fully encapsulated, typically when attached to the heart wall, for example micro-pacemaker, sensors, or pacemaker leads. The same is also observed for vena cava filters, which cannot be removed after long period of implantation of several months. There is therefore a need for a repositionable intravascular or intraluminal device that is less prone to deposit than known devices.

Another issue with known devices is that of the positional stability. Repositionable intraluminal devices are typically anchored at the desired position by means of a number of expandable legs that push on the inner wall of a body lumen or of a vessel. Although these devices have shown remarkable stability in tests, there is a concern that they might wander away of their intended station. Hence, there is a need for a repositionable intravascular or intraluminal device whose position can be stabilized more reliably than the known ones.

Positional stability is particularly important for vascular filters. These devices have the function of capturing and limiting the movements of emboli in the bloodstream. Filters are commonly used in venous system for short time or left permanently. They need a strong fixation to the wall to resist blood pressure and, mostly, have hooked struts or hooked fixations forced inside the wall of the veins, leading sometimes to perforations during implant, permanence, and/or recovery. An implantable device with an improved stability, but not presenting the risks described above, could measure physiological parameters or perform other medical tasks for a short period, and then being recaptured and removed from the vessel or, in long-term applications, it could be explanted if required by an infection or another special medical situation. (ex: Blood pressure monitoring device within pulmonary artery)

### BRIEF SUMMARY OF THE INVENTION

According to the invention, these aims are achieved by means of the object of the appended claims.

### BRIEF DESCRIPTION OF THE DRAWINGS

The invention will be better understood with the aid of the description of an embodiment given by way of example and illustrated by the figures, in which:

FIG. 1 shows an implantable device and a delivery/recovery apparatus according to an aspect of the present invention, the implantable device being in an expanded configuration in a body vessel or lumen;

FIG. 2 represents a known retrievable intraluminal or intravascular device;

FIG. 3 shows a cross-section of the implantable device represented in FIG. 1.

FIG. 4 illustrates the implantable device of the invention in a delivery/recovery apparatus, the implantable device being in a compressed configuration;

FIG. 5 illustrates another aspect of the invention in which the implantable device is anchored by a special positioning element;

FIG. 6 illustrates a variant of the implantable device of FIG. 3;

FIG. 7 illustrates the implantable device of FIG. 4 inside a delivery/recovery apparatus.

FIG. 8 illustrates a variant with a pair of double extended pre-shaped legs for special positioning and safety to avoid migration.

FIG. 9 illustrates another embodiment of the present invention with an extended forked fixation leg.

FIG. 10 illustrates another embodiment of prior art with a sensor or feature on a lateral side with an extended fixation leg

FIG. 11 illustrates the implantable device of FIG. 10 in a compressed state within a catheter

FIG. 12 illustrate another embodiment of a lateral sensor with two fixation struts, an extended leg for safety fixation, and a loop for a recapture.

FIG. 13 illustrates another embodiment of device in FIG. 12 with pre-shaped fixation wires.

FIG. 14 illustrates another variant of the inventive device equipped with a sensor.

### DETAILED DESCRIPTION OF POSSIBLE EMBODIMENTS OF THE INVENTION

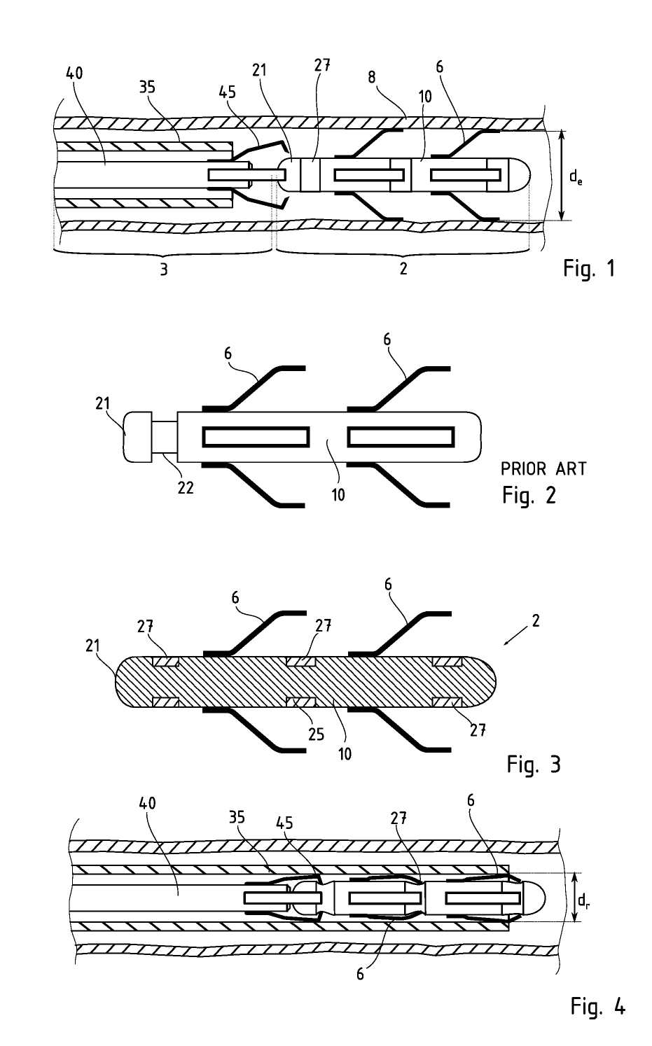

According to a first embodiment of the invention, the implantable device 2 comprises a core 10 of elongated shape, whose dimensions may be determined in order to fit into a specific body vessel or a body lumen. The core 10 is equipped with a plurality of elastic struts or legs 6, which have a natural released configuration, shown in FIGS. 1 and 2, in which the struts extend from the body 10, and can assume, when suitably constrained, a compressed configuration, in which they are almost in full contact with the body 10.

It will be understood that the struts 6 can assume also all intermediate configuration between the fully extended and the fully compressed one and, the overall diameter of the implantable device 2 can have an expanded value dₑ, when the struts 6 are in the free extended configuration, or a compressed value dᵣ when the struts 6 are as close as possible to the core 10, and also all the values comprised between these two extreme limits. It will therefore be clear that the elastic struts 6 allows the implantable device 2 to be positioned in any body lumen or vessel, or in any artificial cavity whose transverse dimensions are comprised between these two extremes. It is also clear how, designing appropriately the number, size, springiness and disposition of the elastic struts 6, the implant of the invention can be position safely also in lumen or vessel whose internal dimensions are irregular, and can also accommodate, within broad limits, movements of contraction and expansion of said lumen or vessels. It is well established that the inner diameter of the vessel can vary due to pulsation, can dilate or retract. A close observation is mandatory to measure the real size of the vessel with awareness of potential expansion or retraction. The struts must be able to adjust and apply enough pressure on the wall to obtain enough friction avoiding a migration risk. Furthermore, the force applied on the lumen walls cannot be too strong to avoid lesion or perforation.

An implantable device structured as above described, with elastic struts extending out of a central core is described in U.S. Pat. No. 7,801,626 in the name of the applicant and hereby incorporated by reference, which also describes how the probe can be inserted, positioned, displaced, or recovered in or from the body, by means of a recovery/positioning device 3 comprising a catheter 40 equipped with a suitable prehensile device 45, designed to interacting with a grasp section 21 of the implant 2.

The same patent also describes several variants of the elastic expandable legs 4. They can involve cantilevered struts as represented here, but also by wire loops, or any other suitable elastic arrangement, and several diagnostic and therapeutic devices added to the implant, like drug release systems or sensors, stimulation or other therapeutic features including, but not limited to: radiation sources, devices for ablation and/or stimulation, position markers for a medical imagery system, for example radiopaque markers visible in a X-ray image, etc.). It is intended that the present invention include likewise any suitable elastic arrangement having a variable dimension capable of assuming a compressed value dᵣ and an expanded value dₑ greater than dᵣ, as well as any possible diagnostic or therapeutic device.

In known devices, as that represented in FIG. 2, the grasp section of the implantable device 2 consists in a round head 21 separated from the body 10 by a narrower neck 22. The flexible fingers 45 of the catheter 40 can be closed, by advancing the sleeve 35, on the head 21, capturing it, or opened by withdrawing the sleeve 35 to release the implant 2. The same sleeve 35 is also used when recovering the implant, to constrain the struts 6 in the compressed configuration.

It is clear that several shapes could be devised for the grasp section. A recurring limitation of the known devices is, however, that a safe grasp requires some sort of cavity, step, or protrusion with which the fingers 45 can interact. This is in fact the function of neck 22. This presents however an obstacle to the flow of body fluids and promotes deposits. An aspect of the present invention consists in providing a suitable cavity for interaction with the prehensile device 45, and fill it with a biocompatible resilient or polymeric material 27, for example medical silicone, to obtain a streamlined core 10 without steps or abrupt changes in traverse dimensions, as represented in FIGS. 1, 3, and 4. In a non-represented variant, the cavities could be filled by a flexible elastic membrane.

The filling or membrane would improve the flow around the device once placed in the vessel. The objective is to achieve a ‘laminar’ or smooth flow around the implanted device without turbulences that could lead to formation of thrombi, particle attachment, coagulation, or tissue growth. Adhesion, coagulation, and tissue growth could furthermore be hindered by a suitable surface treatment.

When the prehensile device 45 closes on the grasp section of the implant, its fingers 45 deform or dig into the soft material, thereby achieving a solid connection with the implant. In a variant, the head 21 and neck 22 could be also made of a soft material and being grasped by compressed by fingers 45.

FIG. 4 illustrates the implantable device of the invention in a delivery/recovery apparatus, the fingers 45 of the catheter 40 close on the grasp section of the implant and, when the catheter is retreated into the sleeve 35, they exert an axial pulling force to the grip 21, and force the implant to assume the compressed value dᵣ of the variable diameter for retrieval or repositioning of the implant 2, or of a part thereof.

A further advantage of providing soft inserts 27 is that it is possible to have lodgements 25 for the flexible struts 6 as well, which allow them to come closer to the core in the compressed configuration, as visible in FIG. 4. In this manner, it is possible to adopt stronger and longer struts, without compromising the minimal value of the compressed diameter.

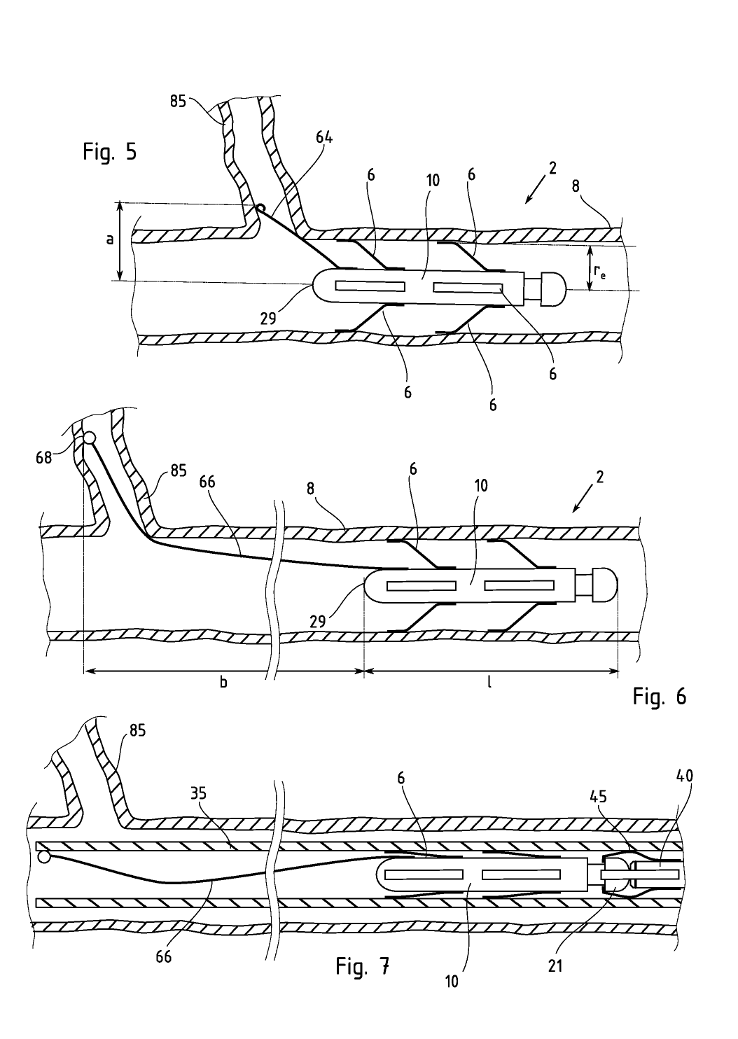

Another aspect of the present invention, that could be combined with the soft inserts described above or adopted independently, is the provision of special elements for positioning and orienting the implant in relation to an anatomic feature, which will now be described in relation to FIGS. 5-7.

FIG. 5 represents a repositionable implant according to this aspect of the invention, located in a vessel 8, for example an artery, in proximity of a branching 85. The implant has a positioning arm or extension 64, specially designed to position in the anatomic feature 85. It can be appreciated that the extension 64 determines a special predetermined relationship of the implant in a relation to the anatomic feature 85, in particular the distance from between the implant and the branching 85 is determined by the size and shape of the arm 64 in relation to the shape of the branching 85, and so is the axial orientation of the implant 2 in relation to the branching.

Importantly, the device of the invention provides an improvement above the known implantable devices in that, while being capable of being retrieved and repositioned, its position relative to a stated anatomical feature, like a branching or a variation of the vessel's diameter, is predetermined, and resists to migration. The positioning arms can be designed to determine the distance of the device from the branching, and/or its orientation relative to the major axis.

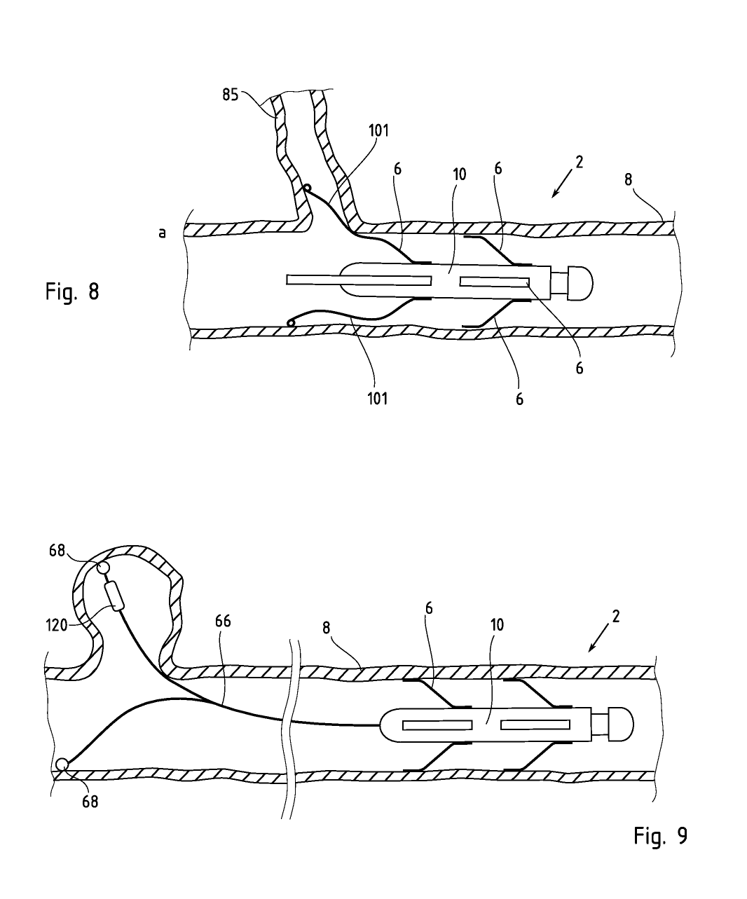

Thanks to this feature, the implant 2 is stably anchored at its intended station in the vessel 8, and cannot be moved by blood circulation or muscular activity, until the times when it is recovered or repositioned. The stabilization function of extension 64 could also be obtained by a combination of several legs (see FIG. 8) or by bifurcated legs (FIG. 9) that effectively anchor the device at a given distance from the bifurcation 85 in the vascular system.

Preferably, the anchoring leg 64 allows increasing the flexibility of the struts 6 whose main function is now centering the implant in the lumen. Accordingly, the force at contact with the walls can be reduced in design, which reduces the risk of lesions.

In general, the length and position of the positioning arm is determined by the anatomy of the feature 85. Preferably, the positioning arm is conformed to extend, in the rest position, at a distance “a” from the axis of the body 10 of the implant 2 and this distance is preferably greater than the radius rₑ of the implant in the extended configuration or, in other words, greater than half of the expanded value dₑ the variable dimension. It is also preferable that the positioning arm 64 extends beyond the extremity of the implant's body 10 that is opposed to the grasp section 21.

The embodiment of FIG. 5 is appropriate for anchoring the implant only in the immediate proximity of a branching or similar feature. It is often the case that the implant must be stationed where no suitable anchoring points are available. FIG. 6 illustrates another variant with a positioning arm 66 that extends beyond the distal extremity 29 of the body 10 by a distance ‘b’ considerably longer than in the previous example. This enables anchoring the implant 2 to an anatomic feature 85 at a greater distance. Preferably, the distance ‘b’ by which the positioning arm extends beyond the implant is greater than the length ‘I’ of the body 10 of the implant itself. The positioning arm 66 is preferably realized with an elastic or, better superelastic material like, for example nickel-titanium (Nitinol) and may be equipped with a blunt atraumatic tip 68. In a variant, the positioning arms (FIG. 8 and FIG. 9) could also carry a sensor or tethered device 120, or be used as communication antenna.

Growth of tissue is mainly observed around the parts in direct contact with walls of lumen. Adherence of particles on the surface directly exposed to flow within the vessel can be decreased by hydrodynamic design and figure surface treatment. (Medication treatment can be used to help avoid thrombosis formation during an invasive monitoring).

FIG. 7 illustrates the configuration of the implant of FIG. 6 in the compressed configuration in the delivery/recovery device comprising the sleeve 35 and the catheter 40. The flexibility of the positioning arm 66 allows compressing it into the sleeve 35, for delivery and/or recovery.

FIG. 8 illustrates the embodiment of a device with extended distal legs. The legs are longer than the proximal set of legs and one of them can be used as lateral fixation within a bifurcation.

FIG. 9 shows another embodiment of device from FIG. 6 with one or more extended legs bifurcating, or forking into any number of sub-branches, for lateral fixation at a crossing of vessel. The design relies on the shape of the legs, rather than on the friction force against the walls to avoid migration or motion of implant. Contact force of strut 6 against wall could be decreased avoiding potential lesion while guaranteeing centering of sensor in vessel. This form of realization is particularly suitable for the monitoring of aneurysms by the in-situ tethered sensor 120.

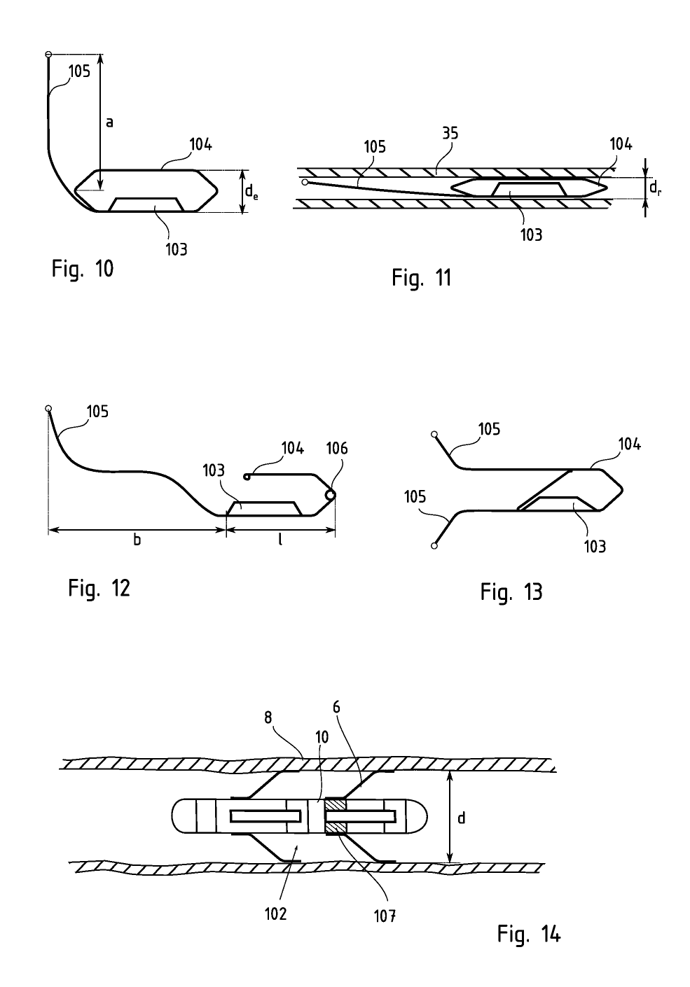

FIG. 10 illustrate an embodiment with the sensor or body 103 fixed on the lateral side and being kept by one or several wire loops 104. A positioning arm 105 for lateral fixation is fixed at the distal part of the sensor and extend distally beyond the body. Several extension arm could be used too. Preferably, the positioning arm 105 extends laterally by a distance ‘a’ that is greater than the expanded diameter de of the implant, in order to engage with a suitable anatomic feature of the lumen in which the device is positioned.

FIG. 11 illustrate the compressed status of the device presented in FIG. 11. The body 103, wire loop 104, and positioning arm 105 are compressed to the diameter dᵣ, corresponding to the inner calibre of the catheter 35. Advantageously, the positioning arm 105 is configured to extend laterally and is adapted for engaging with a branching in the vascular system, or another suitable anatomical feature 85. The device of FIG. 11 is thus stabilized at the intended position by the shape of the positioning arm 105, rather than by the lateral contact force against the walls. Therefore, the risk of damaging the wall 8 of the vessel is greatly reduced.

FIG. 12 illustrates another variant of the implantable device of the invention in which the fixation elements include a first flexible arm 104 essentially lateral to the body 103, and a second flexible arm 105 extending beyond and laterally with respect to the body. Preferably, the positioning arm 105 extends distally beyond the body 103 by a distance ‘b’ that is greater than the length ‘I’ of the implant, in order to engage with a suitable anatomic feature of the lumen in which the device is positioned. The device has also a proximal capture loop 106 for interaction with the catheter prehensile device.

FIG. 13 illustrates another embodiment in which the body of the sensor is positioned laterally in an elastic wire frame 104 that can expand and contract similarly to the devices of FIGS. 10-12, and position is assured by a symmetric pair of distal elastic arms extending beyond the body. The dimensions and shapes of the distal arms are designed to locate the device body 103 at a desired location with respect to an anatomic feature that is in engagement with the elastic arms 105.

Although the devices of FIGS. 10-13 are represented as flat, this is not a limiting feature of the invention that could include any suitable two-dimensional or three-dimensional structure of flexible wire, insofar as it allows an elastic variation of the lateral dimension between the intended values dᵣ and dₑ.

FIG. 14 illustrates a variant of the invention in which the implantable device is equipped with a sensor 107 that is responsive to the deflection of the arms 6. In other terms, since the implantable device has a variable outer diameter that changes in accord with the deflexion of the arms 6, the sensor 107 is responsive to the instant value of this variable dimension.

Thus, the device of FIG. 14 can be regarded as an independent aspect of the present development, which is defined by an implantable device 102 for placement in a body lumen or vessel, for example in a blood vessel 8, comprising: a body 10 with elastic elements 6 having a variable transverse dimension d that corresponds to that of the lumen or vessel 8 in which the device 102 is implanted, and a sensor 107 delivering the instantaneous value of the variable transverse dimension d. Optionally the implantable device 102 can include all the features of the embodiments of the present invention including, but not exclusively: the variable dimension, said variable dimension allowing a compressed value dᵣ for delivery to said body lumen 8 into a catheter and an expanded value dₑ, larger than said compressed value dᵣ, for implantation in said body lumen; a link section, at a first extremity end of the body 10 comprising a grip, for joining said device 102 to a catheter, and for applying an axial force thereto, whereby the device 102 assumes the compressed value dᵣ of said variable dimension and retracts into the catheter for retrieval or repositioning; positioning feature or features extending beyond a second extremity of the body 10 opposed to said first extremity; inserts of a biocompatible resilient or polymeric material adjacent to the grip or to the tip of elastic elements 6, and so on.

In this manner, the implantable device can measure dynamically the inner transverse dimension of the vessel 8, which is clinically significant, and difficult to realize with conventional means. The dimension deduced from arms' deflexion can be combined with the measure of other clinically significant parameters, for example, but not limited to, temperature and pressure. Sensor 107 could also measure flow speed, for example by thermal or ultrasonic flow transducers. Importantly, the simultaneous measure of intravascular pressure and inner dimension allows deducing the instant flow, and elasticity of the vessel's walls.

The sensor or sensors comprised in the implantable device of the invention preferably have a communication interface arranged for transmitting an output, representing one or several clinically significant parameters, to an external unit. Preferably, data transmission is carried out by a suitable wireless communication interface, for example a radio transmitter, a NFC (near field communication) transmitter, or an inductive backscattering circuit. The transmitting unit (not represented in the figures, can be incorporated in the body of the implantable device 2 or be located in another implanted device connected to the implantable device 2 of the invention, for example a subcutaneous device. The communication interface may involve also external dermal patches.

When the implantable device communicates through a radio interface, standardised low-power protocols are preferred for reasons of interoperability and energy management. A possible example is Bluetooth Smart® (also known as Bluetooth Low Energy or BLE).

In many cases, the information gathered by the device sensors will be presented on a suitable display device, like a tablet or a computer, for diagnostic use by clinicians. The invention also includes use cases in which the information is consumed by other medical devices, for example, the implantable device of the invention could be a pressure meter that sends data to a pacemaker, or a chemical sensor interfacing with a drug-delivery system, and so on.

The data needs not be transmitted in real time, but could be logged in a suitable memory and transmitted upon request, or read after the device is explanted.

For what the energy source is concerned, the implantable device of the invention could be equipped by an electric power supply that might rely on autonomous on-board batteries, receive the needed energy from an external source, as an inductive loop or external dermal patches, or harvest energy from body motion or any other suitable source.

### REFERENCE NUMBERS

2 implantable device
3 delivery/recovery apparatus
6 strut, arm
8 wall of body vessel or body lumen
10 body
21 knob, head
22 neck
25 lodgement
27 soft insert
29 distal extremity
35 catheter's sleeve
40 catheter
45 prehensile device, fingers
64 positioning arm
66 positioning arm
68 atraumatic tip
85 branch
101 extended distal leg
102 implantable device
103 body, sensor
104 flexible wire
105 fixation leg
106 loop
120 tethered device
a axial reach of the positioning arm
b extension of the positioning arm
dₑ expanded diameter
dᵣ reduced diameter
l length of the implant
re radius in the extended configuration

1. An implantable device, for placement in a body lumen or vessel, preferably in a blood vessel, comprising: an elongated body with a variable dimension, said variable dimension allowing a compressed value for delivery to said body lumen and an expanded value, larger than said compressed value, for implantation in said body lumen; at a first extremity end of the body comprising a grip, for joining said device to a catheter, and for applying an axial force on said device; said device being arranged for reacting to an axial pulling force to said grip by assuming said compressed value of said variable dimension, for retrieval or repositioning of at least part of said implantable device, wherein the implantable comprises a positioning feature extending beyond a second extremity of the body opposed to said first extremity.

2. The implantable device of the claim 1, wherein the positioning feature comprises a flexible arm extending beyond the second extremity by a length greater than a longitudinal dimension of the body.

3. The implantable device of claim 1, wherein the positioning feature comprises a flexible arm extending radially from the longitudinal axis of the body by a distance greater than half of said expanded value of the variable dimension.

4. The implantable device of claim 1, wherein the positioning feature comprise a flexible forked arm extending beyond the second extremity by a length greater than a longitudinal dimension of the body.

5. The implantable device of claim 1, wherein the positioning device is used as antenna for communication or have a sensor attached thereto.

6. The implantable device of claim 1, wherein the positioning arm includes a blunt atraumatic tip.

7. The implantable device of claim 1, comprising a plurality of elastic struts having a released configuration, in which said elastic struts radially protrude from said elongated body, and a compressed position, in which said elastic struts are closer to said elongated body.

8. The implantable device of claim 1, further including inserts of a biocompatible resilient or polymeric material adjacent to the grip.

9. The implantable device of claim 8, comprising a plurality of elastic struts having a released configuration, in which said elastic struts radially protrude from said elongated body, and a compressed position, in which said elastic struts are closer to said elongated body, and inserts of a biocompatible resilient or polymeric material in correspondence with the tips of the elastic struts in the compressed configuration.

10. The implantable device of claim 1, further including a drug-delivery element, or a sensor, or other instrumented therapeutic device.

11. The implantable device of claim 10, comprising a communication interface for transmitting an output of the sensor to an external unit.

12. The implantable device of any one of claims 10 to 11, comprising an electric power supply.

13. The implantable device of claim 1, with a lateral positioning against the wall of the lumen and with expanding loops or flexible struts, and extended arms for positioning.

14. The implantable device of claim 1, including a sensor responsive to the value of said variable dimension.
